(19) United States
(12) Patent Application Publication
(10) Pub. No.: US 2026/0207937 A1
(43) Pub. Date: Jul. 23, 2026

(54) ELECTRICAL STIMULATION SYSTEM

(71) Applicant: ASKLEON LLC, Thousand Oaks, CA (US)

(72) Inventors: Gary James Sterling, Westlake Village, CA (US); Takeo Usui, Tokyo (JP)

(73) Assignee: ASKLEON LLC, Thousand Oaks, CA (US)

(21) Appl. No.: 19/031,174

(22) Filed: Jan. 17, 2025

(51) Int. Cl. A61N 1/36; A61N 1/04

(52) U.S. Cl. CPC A61N 1/36034; A61N 1/0456; A61N 1/0476; A61N 1/0492

(57) ABSTRACT

An electrical stimulation system, to enable electrical stimulation of nerve tissue in a convenient manner without the need to perform invasive interventional treatments. An electrical stimulation system for electrical stimulation of the vagus nerve leading to the heart located in the neck, comprising: an electrode pad installed on the skin surface and having a convex shape in the direction of the skin depth and having an electrode at the tip of the convex shape; and an electrical stimulation device supplying electrical stimulation energy via a conductor to the electrode of the electrode pad, the electrical stimulation energy is supplied continuously or intermittently.

### FIELD OF THE INVENTION

This invention relates to an electrical stimulation system that provides electrical stimulation to nerve tissue.

### BACKGROUND TECHNOLOGY

Conventionally, stimulation devices that provide electrical stimulation to biological tissues (linear tissues) such as nerve tissues and muscles for treatment are known.

The following are examples of such stimulus-generating devices. Examples of such stimulus generating devices include nerve stimulators, pain relief devices, epilepsy treatment devices, and muscle stimulators.

These stimulation generating devices may be used with electrode leads implanted in the living body, so that the electrode leads that transmit electrical stimulation are in close contact with the stimulation target in the living body. These electrode leads comprise at least one electrode part for applying electrical stimulation to biological tissue or detecting electrical excitation generated in biological tissue, a connection part for electrically connecting with the stimulation generator, and a lead, etc., provided between the electrode part and the stimulation generator to transmit electrical stimulation.

For example, Patent Document 1 is equipped with a cardiac pulse generating means to stimulate the heart, a nerve pulse generating means to stimulate the vagus nerve, a cardiac event detecting means to detect spontaneous cardiac events, and a control means to control the cardiac pulse generating means and the nerve pulse generating means, and this control means is used to control the heart rate at a predetermined rate When the heart rate is less than a predetermined rate, the cardiac pulse generating means is activated to perform cardiac stimulation, and when the heart rate is faster than a predetermined rate, the nerve pulse generating means is activated to perform vagal nerve stimulation. In this cardiac therapy device, a cardiac stimulating electrode is placed within the myocardium or atrium, and a nerve stimulating electrode is placed around the vagus nerve in the neck.

Also disclosed in the second patent document is the percutaneous delivery of a nerve stimulation lead into the patient's internal jugular vein to stimulate the vagus nerve through the internal jugular vein.

### PRIOR ART LITERATURE

### Patent Literature

Patent document 1] JP-A-2004-173790

Patent Document 2] U.S. Pat. No. 8,311,647 Publication

### SUMMARY OF THE INVENTION

### Problem to be Solved by the Invention

However, in such conventional stimulation generators, it is not technically easy to attach the electrode portion of the electrode lead to the linear tissue of the living body, such as nerve tissue. Specifically, nerve tissue is thin and often has blood vessels adjacent to it. When attaching an electrode to nerve tissue, it is necessary to partially detach the tissue that joins the nerve tissue to the blood vessels and attach the electrode portion that provides electrical stimulation to this separated portion. In this process, careful attachment is required to avoid damaging the nerve tissue.

When electrical stimulation is applied by bringing the electrode part into direct contact with the nerve tissue, it is necessary to ensure that the hard electrode part contacts the nerve tissue, which is flexible linear tissue, and thus the nerve tissue must be pressed down to some extent. If the pressing force is too large, the nerve tissue may be compressed, making it impossible to achieve a good therapeutic effect, or the nerve tissue may be damaged.

Furthermore, when an electrode part that stimulates the nerve is implanted in a blood vessel, sufficient measures are considered necessary for means to inhibit thrombus generation and also for damage over time to the vascular endothelium caused by the implantation during the treatment period. In addition, since these prior techniques are considered based on the premise of interventional treatment for the patient, a certain amount of procedural time is required before the nerve is stimulated. For urgent cases, we imagine that sufficient consideration of this time will be required and will be problematic.

The present invention was made in view of the above-mentioned issues and has as its object to provide an electrical stimulation system that can easily perform electrical stimulation to nerve tissue without performing interventional treatment on the patient.

### Means for Solving the Problem

In order to solve the above problems, the electrical stimulation system of the present invention comprises an electrode pad that is placed on the surface of the skin, has a convex shape toward the depth of the skin, and has an electrode at the tip of the convex shape, in order to electrically stimulate the vagus nerve located in the neck that leads to the heart, and an electrical stimulation device that supplies electrical stimulation energy to the electrode of the electrode pad via a conductor, and is characterized in that the electrical stimulation energy can be selected to be supplied continuously or intermittently.

In addition, in the electrical stimulation system of the present invention, it is preferable that the electrode pads are composed of at least one pair, and that biphasic waveform electrical energy is supplied from the electrical stimulation device to stimulate the vagus nerve from at least two or more locations at different times.

In addition, in the electrical stimulation system of the present invention, it is preferable that the electrode pads are configured in at least two pairs, and electrical energy having sine wave waveforms of different frequencies is supplied from the electrical stimulation device, and the vagus nerve is stimulated by interferential stimulation.

### Effects of the Invention

The electrical stimulation system of the present invention has the effect of electrically stimulating the vagus nerve running inside the neck from an electrode pad placed on the surface of the skin of the neck, without the need for interventional treatment on the patient.

### BRIEF DESCRIPTION OF THE DRAWINGS

FIG. 1 A schematic diagram showing the appearance of an electrical stimulation system according to a first embodiment of the present invention when attached to the neck.

FIG. 2 An electrical stimulation system according to the first embodiment.

FIG. 3 A partial cross-sectional view of the side of an electrode pad of the electrical stimulation system according to the first embodiment and a back view from the direction of the attachment surface.

FIG. 4 An explanatory diagram of a neck cross-section of the electrical stimulation system according to the first embodiment.

FIG. 5 A block diagram of an electrical circuit of the electrical stimulation system according to the first embodiment.

FIG. 6 An output pulse waveform diagram of the electrical stimulation system according to the first embodiment.

FIG. 7 An explanatory diagram of the electrical stimulation system according to the first embodiment during continuous stimulation.

FIG. 8 An explanatory diagram of the electrical stimulation system according to the first embodiment during intermittent stimulation.

FIG. 9 An explanatory diagram of heart rate changes due to the electrical stimulation system according to the first embodiment.

FIG. 10 A schematic diagram showing the appearance of an electrical stimulation system according to a second embodiment of the present invention when attached to the neck.

FIG. 11 An electrical stimulation system according to the second embodiment.

FIG. 12 is a partial cross-sectional view of the side of an electrode pad of an electrical stimulation system according to a second embodiment, and a back view from the direction of the attachment surface.

FIG. 13 is an explanatory diagram of a cervical cross-section of an electrical stimulation system according to a second embodiment.

FIG. 14 is an output pulse waveform diagram of an electrical stimulation system according to a second embodiment.

FIG. 15 is an explanatory diagram of an electrical stimulation system according to a second embodiment during continuous stimulation.

FIG. 16 is an explanatory diagram of an electrical stimulation system according to a second embodiment during intermittent stimulation.

FIG. 17 is a schematic diagram showing an electrical stimulation system according to a modified example of the second embodiment of the present invention when attached to the cervical region.

FIG. 18 is an electrical stimulation system according to a modified example of the second embodiment.

FIG. 19 is a block diagram of an electrical circuit of an electrical stimulation system according to a modified example of the second embodiment.

FIG. 20 is an explanatory diagram of a modified example of an electrical stimulation system according to a modified example of the second embodiment during intermittent stimulation.

FIG. 21 is a schematic diagram showing an electrical stimulation system according to a third embodiment of the present invention when attached to the cervical region.

FIG. 22 is an electrical stimulation system according to a third embodiment.

FIG. 23 is an explanatory diagram of a neck cross section of an electrical stimulation system according to a third embodiment.

FIG. 24 is an output pulse waveform diagram of two systems of an electrical stimulation system according to a third embodiment.

FIG. 25 is an explanatory diagram of an electrical stimulation system according to a third embodiment during continuous stimulation.

FIG. 26 is an explanatory diagram of an electrical stimulation system according to a third embodiment during intermittent stimulation.

### DESCRIPTION OF THE EMBODIMENTS

Below, an embodiment of the present invention will be described with reference to the attached drawings. In all drawings, even if the embodiment is different, the same or corresponding parts are given the same reference numerals, and common explanations will be omitted.

### First Embodiment

The electrical stimulation system according to the first embodiment of the present invention will be described.

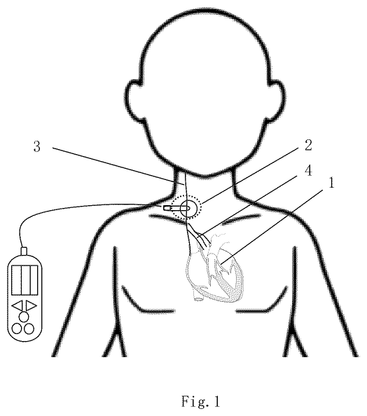

FIG. 1 is a schematic diagram showing the state of the electrical stimulation system according to the first embodiment of the present invention when attached to the neck.

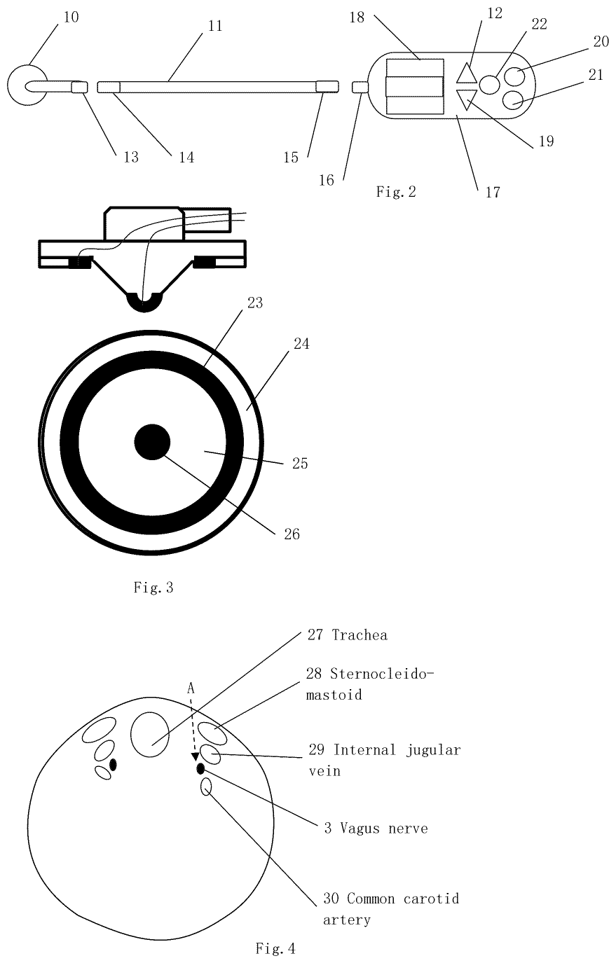

FIG. 2 is the electrical stimulation system according to the first embodiment.

FIG. 3 is a partial cross-sectional view of the side of the electrode pad of the electrical stimulation system according to the first embodiment and a back view from the direction of the attached surface.

FIG. 4 is an explanatory diagram of the neck cross section of the electrical stimulation system according to the first embodiment.

FIG. 5 is a block diagram of the electrical circuit of the electrical stimulation system according to the first embodiment.

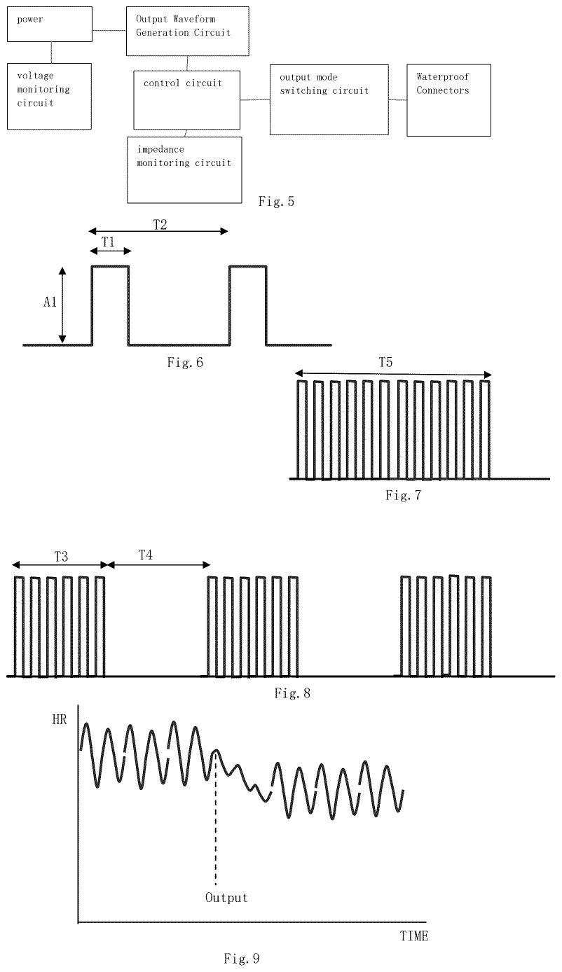

FIG. 6 is an output pulse waveform diagram of the electrical stimulation system according to the first embodiment.

FIG. 7 is an explanatory diagram of the electrical stimulation system according to the first embodiment during continuous stimulation.

FIG. 8 is an explanatory diagram of the electrical stimulation system according to the first embodiment during intermittent stimulation.

FIG. 9 is an explanatory diagram of the heart rate change by the electrical stimulation system according to the first embodiment.

Note that each drawing is a schematic diagram, so the shapes and dimensions are exaggerated (the same applies to the following drawings).

In recent years, in the field of acute myocardial infarction treatment, it has become clear that some moderate and severe patients have a worsening prognosis after reperfusion treatment, and it has become recognized that this leads to chronic heart failure. As a new treatment, it has become known that activating the vagus nerve leading to the heart reduces cardiac load and exerts anti-inflammatory effects and is expected to improve prognosis. For this reason, a nerve stimulator that directly or indirectly applies electronic intervention to the vagus nerve has been considered, and it has become known that it can correct circulatory regulation abnormalities.

The invention is a non-invasive vagus nerve electrical stimulation system designed for therapeutic and medical cardiac applications. The system consists of a convex-shaped electrode pad that is ergonomically designed to conform to the contours of a person's neck, ensuring both comfort and precision in stimulating the branch of the vagus nerve that continues to the heart. The electrode pad is made from flexible, biocompatible materials and is positioned externally on the user's neck, directly over the target nerve area. The electrical stimulation energy is supplied continuously or intermittently.

The electrical stimulation system of the present embodiment can be particularly suitably used for such a treatment of electrically stimulating the vagus nerve 3 near the heart 1.

The electrical stimulation system of this embodiment includes an electrode pad 10, a covered conductor member 11, and an electrical stimulation device 17, As shown in FIGS. 1 and 2.

As shown in FIG. 1, the right vagus nerve 3, which is electrically stimulated in the present invention, branches near the superior vena cava and reaches the heart 1 as a cardiac branch 4. As shown in FIG. 4, in the neck, the right vagus nerve 3 runs behind the right sternocleidomastoideole 28 relative to the trachea 27 and is located between the internal jugular vein 29 and the common carotid artery 30. It is closest to the skin surface near the right brachiocephalic artery. Therefore, the electrode pad 10 of the present invention is attached to the center of the root position 2 of the sternocleidomastoid muscle 28, where electrical stimulation can reach most easily, at position A in FIG. 4. Note that the vagus nerve 3 can be activated by increasing the electrical energy at positions other than A on the neck, but the sternocleidomastoideole 28 and the like are also stimulated, which may increase the burden on the patient.

As shown in FIG. 2,the electrical stimulation system is configured such that electrode pad 10 and electrical stimulation device 17 are connected by covered conductor member 11. Waterproof connectors 13, 14, 15, and 16 are provided at the connection portions, so that electrode pad 10 and electrical stimulation device 17 can be directly connected without using covered conductor member 11.

As shown in FIG. 3, the electrode pad 10 is circular with a diameter of about 30 mm, and the surface to be attached to the skin surface has a cone-shaped support 25 made of nylon resin with a hardness of 50 degrees or less and a convex center. A carbon negative electrode 26 having a diameter of approximately 9 mm is installed at the tip of the cone-shaped support 25, and a ring-shaped carbon positive electrode 23 having an outer diameter of approximately 20 mm and an inner diameter of approximately 15 mm is configured at the base of the cone-shaped support 25. The adhesive gel 24 that is attached to the skin surface is placed around the outer periphery of the adhesive gel 24. The adhesive gel 24 is preferably one that contains a moisturizing agent and/or electrolyte in the polymer matrix and has excellent adhesion, resistance to drying, and electrical conductivity. For example, a suitable product is “Technogel” manufactured by Sekisui Chemical Co., Ltd. In the case of a conductive adhesive gel 24, it may be formed on the negative electrode 26 and the positive electrode 23 as well, which can further improve adhesion to the skin surface and reduce electrical impedance. The height of the cone shape of the support 25 is about 10 mm, and the negative electrode 26 is brought close to the vagus nerve 3 from the skin surface. The electrical energy required to activate the vagus nerve 3 is a negative charge, and by shortening the distance between the negative electrode 26 and the vagus nerve 3, the transmission distance of the electrical energy can be shortened, and the electrical energy required to activate the vagus nerve 3 can be reduced.

The negative electrode 26 and the positive electrode 23 are wired to the waterproof connector 13 via wiring inside the support 25.

The electrical stimulation device 17 of the present invention outputs a monophasic waveform using a constant current method.

As shown in FIG. 5, the electrical stimulation device 17 is composed of a power supply, an output waveform generating circuit, a control circuit, an output mode switching circuit, and a waterproof connector 16. In addition, a voltage monitoring circuit monitors the power supply voltage and detects when the battery is dead. In addition, an impedance monitoring circuit detects peeling of the electrode pads from the skin surface, and breaks and short circuits in the wiring path. When an error is detected, an error indicator light 21 installed on the operation panel flashes.

The device is equipped with a battery that serves as an internal power source, and an output waveform generating circuit generates an electrical stimulation waveform (shape of the waveform) to be supplied to the electrode pad 10. The control circuit sets the magnitude of the electrical energy of the waveform (magnitude of current, pulse width, frequency) based on settings from the operation panel. The output mode switching circuit has the function of switching between continuous stimulation and intermittent stimulation based on settings from the operation panel.

On the operation panel, the selected output mode, current magnitude, pulse width, and frequency are displayed by an LED or LCD 18, and it is also equipped with UP/DOWN buttons 12, 19 for changing each setting, a setting item switching button 22, a decision button 20, and an error warning light 21.

As shown in FIG. 6, the electrical stimulation of the present invention outputs a positive rectangular current waveform, and the current magnitude, pulse width, and frequency can be varied based on the settings on the operation panel. The current magnitude A1 can be varied in 1 mA steps within the range of 1 mA to 40 mA. The pulse width T1 can be varied in 100 μsec steps within the range of 400 μsec to 1000 μsec. The frequency T2 can be varied in 5 Hz steps within the range of 10 Hz to 20 Hz. Since the electrical impedance between the skin surface and the electric pad 10 differs depending on the state of attachment to the patient, it is desirable to be able to vary it for each patient.

For the electrical stimulation to efficiently activate the vagus nerve 3, it is desirable that the frequency T2 is 10 Hz to 20 Hz and the pulse width T1 is 1000 μsec or less. At a frequency T2 of 20 Hz or more, the reaction of the vagus nerve 3 becomes excessive, and the reaction becomes slower due to long-term electrical stimulation. In addition, since the electrical stimulation is from the skin, a pulse width T1 of 400 μsec or less makes it difficult to deliver effective electrical energy to the vagus nerve 3 located deep from the skin surface, and the current magnitude A1 needs to be relatively large, which is undesirable from the viewpoint of muscle fatigue of the sternocleidomastoid muscle 28.

For example, the vagus nerve 3 can be activated under the conditions of a current magnitude A1 of 40 mA, a pulse width T1 of 400 μsec, and a frequency T2 of 20 Hz, and by increasing the pulse width T1, the current magnitude A1 and frequency T2 can be reduced instead.

FIG. 7 shows a case where electrical stimulation is continuously output, and FIG. 8 shows a case where electrical stimulation is intermittently output. As shown in FIG. 9, the heart rate (HR) of an awake patient fluctuates due to respiratory fluctuations and body movements. When electrical stimulation is started and the vagus nerve 3 is activated, acetylcholine is released from the end of the cardiac branch 4 of the vagus nerve 3, which acts on the muscarinic receptors, causing a gradual decrease in the heart rate (HR). For example, when electrical stimulation is started under the conditions of a current magnitude A1 of 40 mA, a pulse width T1 of 400 μsec, and a frequency T2 of 20 Hz, a decrease in the average heart rate including respiratory fluctuations of about 3% to 5% can be achieved.

Therefore, it is possible to determine whether the electrode pad 10 is attached in an appropriate position by monitoring the patient's heart rate (HR) using an external electrocardiograma like.

On the other hand, because of changes in heart rate HR caused by respiratory fluctuations, it is difficult to determine whether or not the heart rate HR has decreased due to electrical stimulation by applying electrical stimulation for a brief period of time (several seconds). For this reason, it is easier for the operator of the electrical stimulation system to check whether the electrode pad 10 is attached to an appropriate position by applying continuous stimulation rather than intermittent stimulation. When starting treatment, it is desirable to adjust the attachment position of the electrode pad 10 by continuous stimulation, and then start intermittent stimulation after confirming that it is in the appropriate position.

In the present invention, since electrical energy is also transmitted to the sternocleidomastoideole 28, in order to avoid muscle fatigue due to long-term continuous stimulation, the continuous stimulation can be stopped at the operator's discretion but has a function to automatically stop after 120 seconds. As shown in FIG. 8, from the viewpoint of muscle fatigue of the sternocleidomastoideole 28, the intermittent stimulation is configured to repeat an output time T3 of 10 seconds and an output stop time T4 of 50 seconds until the end of treatment.

In patients who have suffered acute myocardial infarction and undergone reperfusion therapy, troponin, an inflammatory marker for the heart 4 (released from the heart into the circulating blood when the myocardium is damaged; a biomarker used as an indicator of myocardial damage), shows high values for about three days. Therefore, it is advisable to perform intermittent stimulation for at least one day and continue treatment for three days if the patient can tolerate it.

When the vagus nerve 3 is activated by electrical stimulation, acetylcholine is released from the end of the cardiac branch 4, which acts on muscarinic receptors to reduce the heart rate and reduce the load on the heart. Acetylcholine also acts on nicotinic receptors and exerts an anti-inflammatory effect. In some patients, there is a concern that an excessive reaction after reperfusion treatment (in other words, reperfusion injury) may cause the myocardial necrotic area to expand, leading to cardiac remodeling. In the present invention, by activating the vagus nerve 3 by electrical stimulation, the two effects mentioned above (reducing cardiac load and anti-inflammatory effect) are exerted, and it is expected that excessive responses will be suppressed, and cardiac remodeling will be inhibited.

As a method for electrically activating the vagus nerve 3, a method of wrapping an implantable electrode around the vagus nerve 3 and directly applying electrical stimulation, and a method of indirectly applying electrical stimulation from within the blood vessel to the vagus nerve 3 running near the blood vessel by an interventional technique have been proposed. These methods cannot be applied to acute illnesses because it takes several days for the nerve and the electrode to adhere and stabilize, and they require an X-ray fluoroscopy device to place the device in the blood vessel, and special measures against the occurrence of thrombosis (application of antithrombotic materials and postoperative management with anticoagulants), so the burden on patients associated with interventional treatment on the human body is expected to be a concern.

The present invention is a non-invasive treatment, and since it is a quite simple method that involves only attaching the electrode pad 10 to the skin surface, it has the advantage that it is particularly suitable for acute cases and treatment can be started easily. Therefore, it is possible to stimulate the vagus nerve (start treatment) immediately after the patient arrives at the hospital and is definitively diagnosed with acute myocardial infarction.

Also, unlike drug therapy, by varying the electrical energy, the load reduction effect on the heart 1 and the anti-inflammatory effect can be digitally varied, making it easier to provide optimal treatment tailored to the patient.

In addition, the electrical stimulation device 17 may be equipped with a wireless communication function, and the stimulation conditions, output mode, and stimulation output start and stop may be controlled from an external terminal such as a PC. Furthermore, it is understood that additional functions such as patient ID registration, setting of the treatment end time, and history management of patient information such as storage of operation record logs may be added.

The device's wireless option enhances user convenience and mobility, eliminating the need for cumbersome cables during treatment. The system can be controlled manually or via a mobile application, allowing users or healthcare providers to customize the stimulation sessions easily.

This vagus nerve stimulation system provides a non-invasive alternative to traditional surgical methods, reducing risks and recovery times while offering a versatile tool for managing reperfusion and inflammation, reducing the potential for heart failure, cardiac arrhythmias, various neurological and physiological conditions such as epilepsy, depression, and chronic pain.

### Second Embodiment

Next, an electrical stimulation system according to a second embodiment of the present invention will be described.

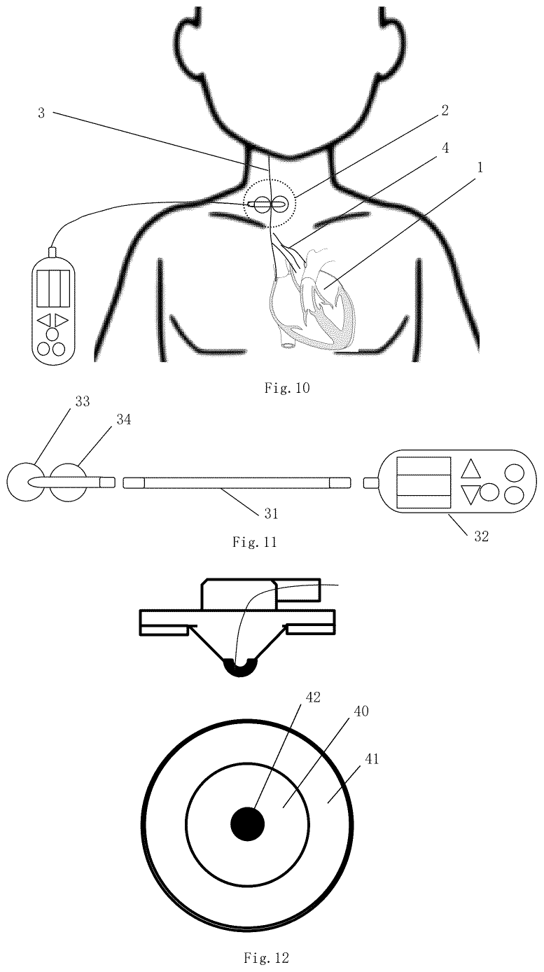

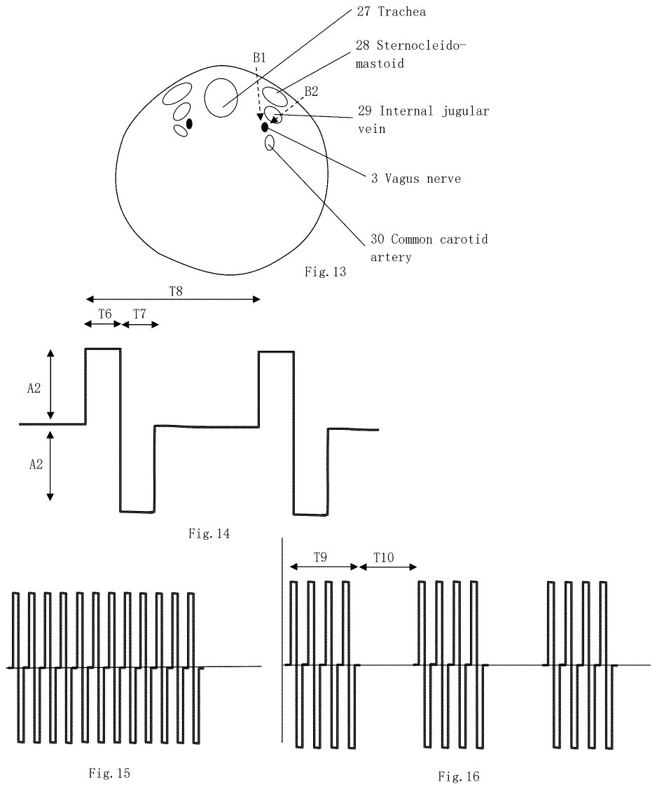

FIG. 10 is a schematic diagram showing the state of an electrical stimulation system according to a second embodiment of the present invention when attached to the neck. FIG. 11 is an electrical stimulation system according to the second embodiment. FIG. 12 is a partial cross-sectional view of the side of an electrode pad of the electrical stimulation system according to the second embodiment and a back view from the direction of the attachment surface. FIG. 13 is an explanatory diagram of a neck cross section of the electrical stimulation system according to the second embodiment. FIG. 14 is an output pulse waveform diagram of the electrical stimulation system according to the second embodiment. FIG. 15 is an explanatory diagram of the electrical stimulation system according to the second embodiment during continuous stimulation. FIG. 16 is an explanatory diagram of the electrical stimulation system according to the second embodiment during intermittent stimulation.

Note that explanations of parts that are the same as those of the first embodiment will be omitted.

As shown in FIGS. 10 and 11, the electrical stimulation system of the present embodiment includes an electrode pad 30, a covered conductor member 31, and an electrical stimulation device 32.

As shown in FIG. 10, the electrode pads 33, 34 of the present invention are attached to both sides of the base 2 of the sternocleidomastoideole 28 where electrical stimulation is most easily accessible, at positions B1 and B2 in FIG. 13.

As shown in FIG. 11, the electrical stimulation system includes a pair of electrode pads 33, 34 and an electrical stimulation device 32 connected together by a covered conductor member 31.

As shown in FIG. 12, the electrode pads 33 and 34 are circular with a diameter of about 30 mm, and the surface to be attached to the skin surface has a cone-shaped support 40 made of silicone resin with a hardness of 25 degrees and a convex center. A carbon electrode 42 with a diameter of about 9 mm is installed at the tip of the cone shape of the support 40, and an adhesive gel 41 that reliably adheres to the skin surface is installed at the base of the cone-shaped support 40. The adhesive gel 41 is the same as in the first embodiment. The height of the cone shape of the support 40 is the same as in the first embodiment, but since it is made of a soft silicone resin support 40, it can be appropriately deformed to the unevenness of the skin surface at the attachment location 2, and the electrode 42 can be prevented from floating up. In addition, the electrode pads 33 and 34 are configured as a pair, and the negative and positive wiring of the internal wiring from the electrical stimulation device 32 is connected to the electrodes of the electrode pads 33 and 34, respectively. For example, the negative wiring is connected to electrode pad 33 on the central side of the base of the sternocleidomastoideole (position B1 in FIG. 13), and the positive wiring is connected to electrode pad 34 on the right arm side of the base of the sternocleidomastoideole (position B2 in FIG. 13).

The electrical stimulation device 32 of the present invention outputs a constant current biphasic waveform.

As in the first embodiment, the electrical stimulation device 32 includes a power supply, an output waveform generating circuit, a control circuit, an output mode switching circuit, and a waterproof connector.

As shown in FIG. 14, the electrical stimulation of the present invention outputs a biphasic current waveform, and the current magnitude A2, pulse widths T6, T7, and frequency T8 can be varied based on the settings on the operation panel. The current magnitude A2 can be varied in 1 mA steps within the range of 1 mA to 40 mA. The pulse widths T6 and T7 can be varied in 100 μsec steps within the range of 400 μsec to 1000 μsec. The frequency T8 can be varied in 5 Hz steps within the range of 10 Hz to 20 Hz. Since the electrical impedance between the skin surface and the electric pads 33 and 34 differs depending on the state of attachment to the patient, it is desirable to be able to vary it for each patient.

FIG. 15 shows a case where electrical stimulation is continuously output, and FIG. 16 shows a case where electrical stimulation is intermittently output. As in the first embodiment, due to changes in heart rate caused by respiratory fluctuations, it is difficult to determine whether the heart rate is reduced by electrical stimulation by applying electrical stimulation for a brief period of time (several seconds). For this reason, it is easier for the operator of the electrical stimulation system to check whether the electrode pads 33, 34 are attached in the appropriate position by applying continuous stimulation rather than intermittent stimulation. At the start of treatment, the attachment positions of the electrode pads 33, 34 are adjusted by continuous stimulation, and after it is confirmed that they are in the appropriate position, intermittent stimulation is started.

In the present invention, a biphasic waveform is output to a pair of electrode pads 33 and 34.

As vagus nerve 3 is activated by a negative charge, for pulse T6 of the positive component of the biphasic waveform, vagus nerve 3 is stimulated with electrode pad 33 installed at position B1 in FIG. 13. For pulse T7 of the negative component of the biphasic waveform, the vagus nerve 3 is stimulated at electrode pad 34, located at position B2 in FIG. 13. Compared to the first embodiment, twice the electrical energy can be applied to the vagus nerve 3, so the electrical energy to be set can be smaller than that of the first embodiment.

In addition, since the magnitude of the current can be reduced, the patient feels less uncomfortable, and the output time T9 of the intermittent stimulation can be set longer. The output time T9 of 20 seconds and the output stop time T10 of 40 seconds are repeated until the end of treatment. For example, when electrical stimulation is started under conditions of a current magnitude of 20 mA, a pulse width of 400 μsec, and a frequency of 20 Hz, a reduction in the average heart rate, including respiratory fluctuations, of approximately 5% can be achieved.

This can further reduce muscle fatigue of the sternocleidomastoideole 28 caused by long-term electrical stimulation. Furthermore, the number of positions at which electrical stimulation is applied is now two, which can reduce the number of times that the attachment positions of the electrode pads 33, 34 need to be adjusted.

This embodiment can also achieve the same actions and effects as the first embodiment. Intermittent electrical stimulation for one to three days reduces the load on the heart and exerts an anti-inflammatory effect, making it possible to suppress reperfusion injury after reperfusion treatment.

### Modification

Next, an electrical stimulation system according to a modification of the second embodiment of the present invention will be described.

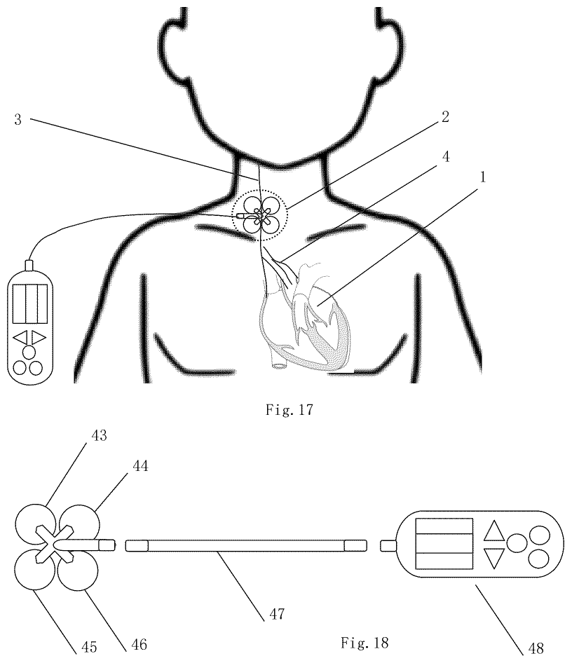

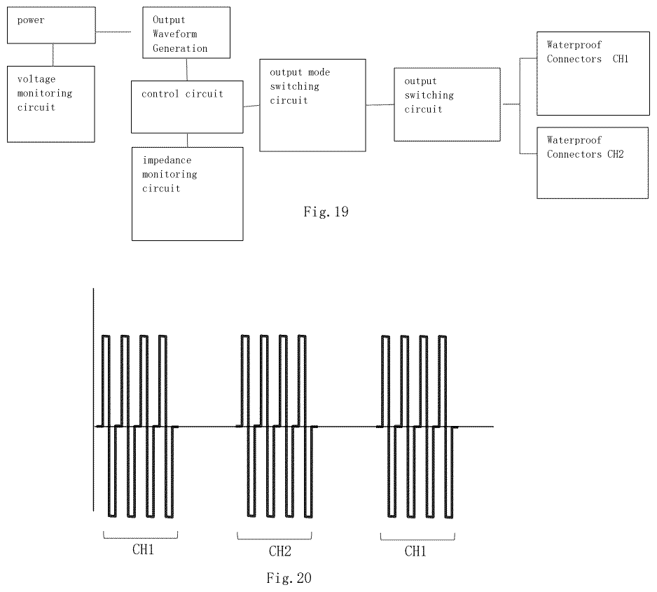

FIG. 17 is a schematic diagram showing an electrical stimulation system according to a modification of the second embodiment of the present invention when attached to the neck. FIG. 18 is an electrical stimulation system according to a modification of the second embodiment. FIG. 19 is a block diagram of the electrical circuit of the electrical stimulation system according to the modification of the second embodiment. FIG. 20 is an explanatory diagram of a modification of the electrical stimulation system according to the modification of the second embodiment during intermittent stimulation.

Only the parts that differ from the second embodiment will be described.

As shown in FIG. 17, electrode pads 43, 44, 45, and 46 of the present invention are attached to both sides of the base 2 of the sternocleidomastoideole 28 where electrical stimulation is most easily accessible.

As shown in FIG. 18, the electrical stimulation system connects two pairs of electrode pads 43, 44, 45, 46 and an electrical stimulation device 48 with a covered conductor member 47.

Note that the electrode pads 43, 44, 45, 46 are the same as those in the second embodiment, but are configured in two pairs, and the internal wiring is separated for the two negative electrodes 43, 44 and the two positive electrodes 45, 46.

The electrical stimulation device 48 of this modified example outputs two systems of constant current biphasic waveforms. As shown in FIG. 19, an output destination switching circuit is added to the electrical stimulation device 48 and wiring on the CH1 side and wiring on the CH2 side are incorporated into the waterproof connector. As with the second embodiment, the current magnitude A2, pulse widths T6, T7, and frequency T8 can be set on the operation panel.

The coated conductor member 47 also has two systems of internal wiring configured to match the two systems of output of the electrical stimulation device 48.

In this modification, the vagus nerve 3 can be activated using two pairs of electrode pads 43, 44, 45, and 46. Since the vagus nerve 3 is activated by a negative charge, the vagus nerve 3 is stimulated by the electrode pads 43 and 44 in response to a pulse T6 of a positive component of a biphasic waveform. The vagus nerve 3 is stimulated by the electrode pads 45 and 46 in response to a pulse T7 of a negative component of a biphasic waveform. Since four times as much electrical energy can be applied to the vagus nerve 3 as in the first embodiment, the electrical energy to be set can be smaller than in the first embodiment.

FIG. 20 shows a modified example of intermittent electrical stimulation.

The output destination of the biphasic waveform can be switched between electrode pads 43 and 46 of CH1 and electrode pads 44 and 45 of CH2 every output time T9 by the output destination switching circuit.

If the vagus nerve 3 at the same position is stimulated for an extended period of time, the reaction of the vagus nerve 3 to the electrical stimulation becomes slower. Specifically, if the same electrical energy is applied continuously, the decrease in heart rate becomes smaller. In this case, the operator of the electrical stimulation system must try to increase the electrical energy within the range that the patient can tolerate. In this modified example, the stimulation position of the vagus nerve 3 is shifted to the positions of electrode pads 43 and 46 of CH1 and electrode pads 44 and 45 of CH2, thereby reducing the phenomenon of the reaction of the vagus nerve 3 becoming slower.

In this modified example, muscle fatigue of sternocleidomastoideole 28 caused by electrical stimulation can be further reduced. Electrical stimulation is also performed at four locations, further reducing the number of times the attachment positions of electrode pads 43, 44, 45, and 46 need to be adjusted. An output destination switching circuit has been added within electrical stimulation device 48, so electrode pads 43 and 46 on the CH1 side and electrode pads 44 and 45 on the CH2 side can also be stimulated independently.

The electrical stimulation system contains electrodes that deliver programmable, biphasic controlled electrical impulses to the vagus nerve.

Depending on the configuration, the electrical stimulation system connects wirelessly or via wires to an external electrical stimulator. The stimulator device is programmable and capable of generating multiple electrical outputs with adjustable parameters such as intensity, frequency, and waveform to accommodate a range of therapeutic needs and patient-specific requirements.

### Third Embodiment

Next, an electrical stimulation system according to a third embodiment of the present invention will be described.

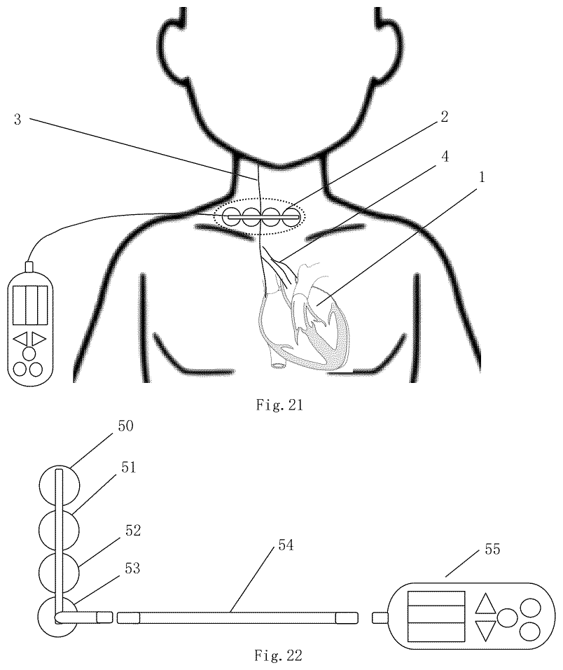

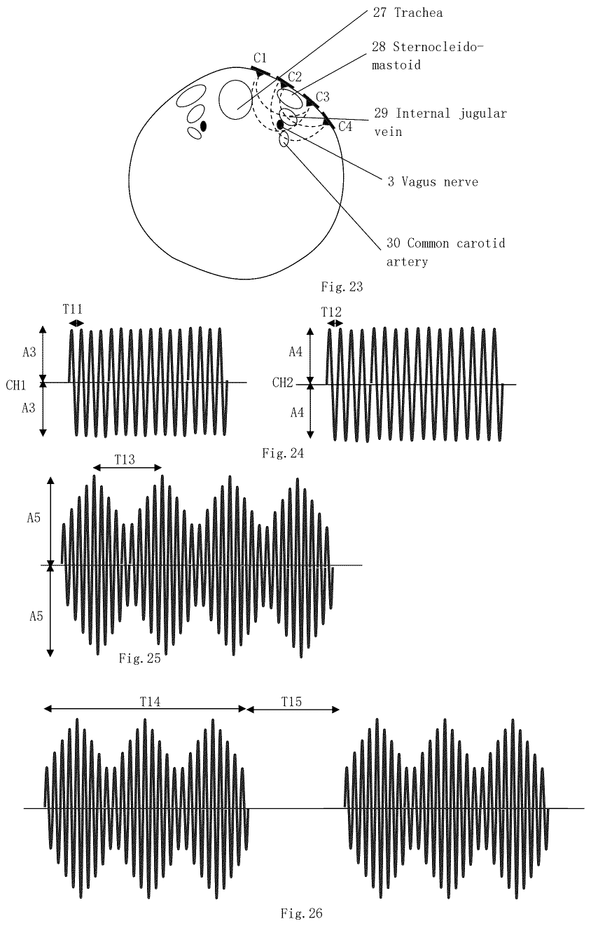

FIG. 21 is a schematic diagram showing an electrical stimulation system according to a third embodiment of the present invention when attached to the neck. FIG. 22 is an electrical stimulation system according to the third embodiment. FIG. 23 is an explanatory diagram of a neck cross section of the electrical stimulation system according to the third embodiment. FIG. 24 is an output pulse waveform diagram of two systems of the electrical stimulation system according to the third embodiment. FIG. 25 is an explanatory diagram of the electrical stimulation system according to the third embodiment during continuous stimulation. FIG. 26 is an explanatory diagram of the electrical stimulation system according to the third embodiment during intermittent stimulation.

As shown in FIGS. 21 and 22, the electrical stimulation system of the present embodiment includes electrode pads 50, 51, 52, and 53, a covered conductor member 54, and an electrical stimulation device 55.

As shown in FIG. 21, electrode pads 50, 51, 52, 53 of the present invention are attached to both sides of the base 2 of the sternocleidomastoideole 28 where electrical stimulation is most easily accessible, at positions C1, C2, C3 and C4 in FIG. 23.

As shown in FIG. 22, the electrical stimulation system connects two pairs of electrode pads 50, 51, 52, 53 and an electrical stimulation device 55 with a covered conductor member 54. Note that the electrode pads 50, 51, 52, 53 are the same as those in the second embodiment, but are configured in two pairs, and the internal wiring is separated for the two negative electrode pads 51, 52 and the two positive electrode pads 50, 53.

The electrical stimulation device 55 of this embodiment outputs two constant current sine wave waveforms.

The electrical stimulation device 55 is configured with a power supply, an output waveform generating circuit, a control circuit, an output mode switching circuit, and a waterproof connector. The CH1 side wiring and the CH2 side wiring are incorporated into the waterproof connector.

The device is equipped with a battery as an internal power source, and an output waveform generating circuit generates an electrical stimulation waveform (sine wave) to be supplied to electrode pads 50, 51, 52, and 53. The control circuit sets the magnitude of the electrical energy of the waveform (the magnitude A3 of the CH1 current, the magnitude A4 of the CH2 current, and the frequency T13) based on the settings from the operation panel. The output mode switching circuit has the function of switching between continuous stimulation and intermittent stimulation based on the settings from the operation panel.

As shown in FIG. 24, the electrical stimulation of the present invention outputs two sinusoidal current waveforms, and the current magnitudes A3, A4 and frequency T13 of each system can be varied based on the settings on the operation panel. The current magnitudes A3 and A4 can be varied in 0.5 mA steps in the range of 1 mA to 5 mA. The frequency T13 can be varied in 1 Hz steps in the range of 10 Hz to 20 Hz.

In this embodiment, in order to selectively stimulate only the deep area, two types of electrical stimulation are transmitted to the skin surface from the electrode pads 50, 51, 52, and 53, and interferential stimulation can be performed in the deep area. As shown in FIG. 23, interference waves are generated in the deep area by superimposing two sine wave currents of slightly different frequencies T11 and T12. In addition, the generation position of the interference waves generated in the deep area can be changed by adjusting the magnitudes A3 and A4 of the currents and the positions of the electrodes.

The frequency T11 of the sine wave actually output to CH1 is 2000 Hz, but the frequency T12 of the sine wave output to CH2 is 2000 Hz plus the frequency T13 set on the operation panel. For example, if frequency T13 is set to 10 Hz, a 2000 Hz sine wave will be output to CH1 and a 2010 Hz sine wave to CH2.

The electrical impedance of the skin surface for frequencies T13 of 10 Hz to 20 Hz as in the first embodiment is several thousand Ω, but for frequencies T11 and T12 of about 2000 Hz, the electrical impedance drops to several hundred Ω. In this embodiment, the frequencies T11 and T12 of the electrical stimulation are increased to lower the skin resistance and reduce the required current magnitudes A3 and A4. In addition, because the frequencies T11 and T12 are high, the reaction of the sternocleidomastoideole 28 is suppressed, muscle fatigue is suppressed, and the discomfort felt by the patient due to the electrical stimulation can be reduced.

The electrode pads 50, 51, 52, and 53 are the same as those in the second embodiment, but two pairs of electrode pads 50, 51, 52, and 53 are arranged linearly. As shown in FIG. 23, the positive electrode 50 of CH2 is arranged on C1, the negative electrode 51 of CH1 is arranged on C2, the negative electrode 52 of CH2 is arranged on C3, and the positive electrode 53 of CH1 is arranged on C4. The dotted lines in the figure indicate the flow of current, and the vagus nerve 3 is located at the position where the currents of CH1 and CH2 intersect. The electrode pads 51 and 52 located close to the vagus nerve 3 are configured as the negative side.

Slightly different sine wave waveforms as shown in FIG. 24 are output to the respective electrode pads 50, 51, 52, and 53. As a result, an interference wave of frequency T13 set from the operation panel as shown in FIG. 25 is generated, and the vagus nerve 3 located deep inside can be stimulated with the set frequency T13.

It is known that when electrodes are directly wrapped around the vagus nerve 3, activation is possible with a current magnitude of 3 mA or less. In this embodiment, the current magnitude A5 at the deep part is increased by the interference wave, so that the vagus nerve 3 can be activated even with the current magnitudes A3 and A4 set to 5 mA or less. Since the current magnitudes A3 and A4 are also small on the skin surface where the electrode pads 50, 51, 52, and 53 are attached, the reaction of the sternocleidomastoideole 28 is small, and the discomfort felt by the patient from the electrical stimulation is significantly reduced.

FIG. 25 shows a case where electrical stimulation is set to continuous output, and FIG. 26 shows a case where electrical stimulation is set to intermittent output. As in the first embodiment, due to changes in heart rate caused by respiratory fluctuations, it is difficult to determine whether the heart rate is reduced by electrical stimulation by applying electrical stimulation for a brief period of time (several seconds). For this reason, it is easier for an operator of the electrical stimulation system to check whether the electrode pads 50, 51, 52, 53 are attached to appropriate positions by applying continuous stimulation rather than intermittent stimulation. At the start of treatment, the attachment positions of the electrode pads 50, 51, 52, 53 are adjusted by continuous stimulation, and after it is confirmed that they are in the appropriate positions, intermittent stimulation is started.

In addition, in this embodiment, because of the interferential stimulation, the magnitudes A3 and A4 of the currents in CH1 and CH2 can be changed, respectively, to slightly adjust the deep position where the strongest interference waves are generated. In this case, there is no need to adjust the attachment positions of the electrode pads 50, 51, 52, and 53, making it possible to shorten the time to start treatment.

In this embodiment, muscle fatigue due to continuous stimulation for an extended period of time is unlikely to occur, and intermittent stimulation is likely to be tolerated by the patient even with an output time T14 of 30 seconds and an output stop time T15 of 20 seconds.

This embodiment can also achieve the same actions and effects as the first embodiment. Since the discomfort caused by electrical stimulation is further reduced, the treatment period can be extended to three days or more. Therefore, the effect of reducing the load on the heart and the anti-inflammatory effect can be maintained for a longer period, and reperfusion injury after reperfusion treatment can be further suppressed.

Furthermore, all of the components described in the above embodiments and modifications can be implemented in different combinations or deleted within the scope of the technical concept of the present invention.

### EXPLANATION OF SYMBOLS

1 Heart
2 Root of sternocleidomastoideole
3 Vagus nerve
4 Cardiac branch
10 Electrode pad
11 Covered conductor
12 UP button
13 Waterproof connector
14 Waterproof connector
15 Waterproof connector
16 Waterproof connector
17 Electrical stimulator
18 LED or LCD
19 DOWN button
20 Confirm button.
21 Error warning light
22 Setting item switching button
23 Positive electrode
24 Adhesive gel
25 Support
26 Negative electrode
27 Trachea
28 Sternocleidomastoideole
29 Internal jugular vein
30 Common carotid artery
31 Covered conductor material
32 Electrical stimulation device
33 Electrode pad
34 Electrode pad
40 Support
41 Adhesive gel
42 Electrode
43 Electrode pad
44 Electrode pad
45 Electrode pad
46 Electrode pad
47 Covered conductor material
48 Electrical stimulation device
50 Electrode pad
51 Electrode pad
52 Electrode pad
53 Electrode pad
54 Covered conductor material
55 Electrical stimulation device

1. An electrical stimulation system for electrically stimulating the vagus nerve located in the neck leading to the heart, comprising:
at least one electrode pad placed on the surface of the skin, convex toward the depth of the skin, and having a first electrode at the tip of the convex shape;
and an electrical stimulation device electrically connected to the electrode of the at least one electrode pad to supply electrical stimulation energy;
the electrical stimulation system is characterized in that the electrical stimulation energy includes at least one of a continuously supplied electrical stimulation energy and an intermittently supplied electrical stimulation energy.

2.-5. (canceled)

6. The electrical stimulation system according to claim 1, characterized in that the electrode pads are composed of at least one pair or two pairs, and the electrical stimulation energy is electrical energy with a sinusoidal waveform of different frequencies supplied to the position of the electrode.

7. The electrical stimulation system according to claim 6, characterized in that the magnitude of the constant current with the sinusoidal waveform is variable in 0.5 mA steps in the range of 1 mA to 5 mA, and the frequency is variable in 1 Hz steps in the range of 10 Hz to 20 Hz.
